SEQUENCE LISTING (29 sequences)

SEQ ID NO: 1 - DNA, 22 bp, synthetic construct
  source 1..22: mol_type = other DNA
gaagagggagcaattgttggcg

SEQ ID NO: 2 - DNA, 27 bp, synthetic construct
  source 1..27: mol_type = other DNA
cagagactcgaactgtgttatcattcc

SEQ ID NO: 3 - DNA, 28 bp, synthetic construct
  source 1..28: mol_type = other DNA
tattggtctcagggagcgaaagcaggtc

SEQ ID NO: 4 - DNA, 20 bp, synthetic construct
  source 1..20: mol_type = other DNA
tcctcttgtgaaaataccat

SEQ ID NO: 5 - DNA, 20 bp, synthetic construct
  source 1..20: mol_type = other DNA
tatgaagagttcacaatggt

SEQ ID NO: 6 - DNA, 34 bp, synthetic construct
  source 1..34: mol_type = other DNA
atatggtctcgtattagtagaaacaaggtcgttt

SEQ ID NO: 7 - DNA, 28 bp, synthetic construct
  source 1..28: mol_type = other DNA
tattcgtctcagggagcgaaagcaggca

SEQ ID NO: 8 - DNA, 20 bp, synthetic construct
  source 1..20: mol_type = other DNA
ttgaacatgcccatcatcat

SEQ ID NO: 9 - DNA, 19 bp, synthetic construct
  source 1..19: mol_type = other DNA
aaatacctgcagaaatgct

SEQ ID NO: 10 - DNA, 33 bp, synthetic construct
  source 1..33: mol_type = other DNA
atatcgtctcgtattagtagaaacaaggcattt

SEQ ID NO: 11 - DNA, 29 bp, synthetic construct
  source 1..29: mol_type = other DNA
tattcgtctcagggagcgaaagcaggtac

SEQ ID NO: 12 - DNA, 23 bp, synthetic construct
  source 1..23: mol_type = other DNA
aactcattctgaatccaacttgc

SEQ ID NO: 13 - DNA, 23 bp, synthetic construct
  source 1..23: mol_type = other DNA
ggaaggaacccaatgttgttaaa

SEQ ID NO: 14 - DNA, 33 bp, synthetic construct
  source 1..33: mol_type = other DNA
atatcgtctcgtattagtagaaacaaggtactt

SEQ ID NO: 15 - DNA, 28 bp, synthetic construct
  source 1..28: mol_type = other DNA
tattcgtctcagggagcaaaagcagggg

SEQ ID NO: 16 - DNA, 22 bp, synthetic construct
  source 1..22: mol_type = other DNA
aatggctccaaatagacctctg

SEQ ID NO: 17 - DNA, 23 bp, synthetic construct
  source 1..23: mol_type = other DNA
gggaggatgaactattactggac

SEQ ID NO: 18 - DNA, 29 bp, synthetic construct
  source 1..29: mol_type = other DNA
tattcgtctcagggagcaaaagcagggta

SEQ ID NO: 19 - DNA, 23 bp, synthetic construct
  source 1..23: mol_type = other DNA
ctattccgactagagagtatccc

SEQ ID NO: 20 - DNA, 22 bp, synthetic construct
  source 1..22: mol_type = other DNA
tcaatgatcggaacttctggag

SEQ ID NO: 21 - DNA, 36 bp, synthetic construct
  source 1..36: mol_type = other DNA
atatcgtctcgtattagtagaaacaagggtattttt

SEQ ID NO: 22 - DNA, 29 bp, synthetic construct
  source 1..29: mol_type = other DNA
tattggtctcagggagcaaaagcaggagt

SEQ ID NO: 23 - DNA, 20 bp, synthetic construct
  source 1..20: mol_type = other DNA
tctctgcacacacacatcac

SEQ ID NO: 24 - DNA, 20 bp, synthetic construct
  source 1..20: mol_type = other DNA
ggctggctaacaatcggaat

SEQ ID NO: 25 - DNA, 36 bp, synthetic construct
  source 1..36: mol_type = other DNA
atatggtctcgtattagtagaaacaaggagtttttt

SEQ ID NO: 26 - DNA, 29 bp, synthetic construct
  source 1..29: mol_type = other DNA
tattcgtctcagggagcaaaagcaggtag

SEQ ID NO: 27 - DNA, 36 bp, synthetic construct
  source 1..36: mol_type = other DNA
atatcgtctcgtattagtagaaacaaggtagttttt

SEQ ID NO: 28 - DNA, 29 bp, synthetic construct
  source 1..29: mol_type = other DNA
tattcgtctcagggagcaaaagcagggtg

SEQ ID NO: 29 - DNA, 35 bp, synthetic construct
  source 1..35: mol_type = other DNA
atatcgtctcgtattagtagaaacaagggtgtttt

(19) United States
(12) Patent Application Publication
(10) Pub. No.: US 2024/0301517 A1
(43) Pub. Date: Sep. 12, 2024

(54) A MICROFLUIDIC PIPELINE FOR ISOLATION AND ANALYSIS OF SINGLE VIRUSES

(71) Applicants: Versitech Limited, Hong Kong (CN); Advanced Biomedical Instrumentation Centre Limited, Hong Kong (CN); Centre for Immunology & Infection Limited, Hong Kong (CN)

(72) Inventors: Ho Cheung SHUM, Hong Kong (CN); Lit Man POON, Hong Kong (CN); Lang NAN, Hong Kong (CN); Man Yuk Alison LAI, Hong Kong (CN)

(21) Appl. No.: 18/590,291

(22) Filed: Feb. 28, 2024

Related U.S. Application Data
(60) Provisional application No. 63/488,802, filed on Mar. 7, 2023.

(51) Int. Cl. C12Q 1/70; B01L 3/00; C12N 15/10; C12Q 1/6874; G01N 21/64

(52) U.S. Cl. CPC C12Q 1/70; B01L 3/502784; C12N 15/1096; C12Q 1/6874; G01N 21/6486; B01L 2200/0636; B01L 2300/0663; B01L 2300/0809; B01L 2400/0487

(57) ABSTRACT

The subject invention pertains to a microfluidic pipeline which enables isolation and analysis of single viruses. Single viruses are encapsulated and manipulated within microfluidic droplets. The subject invention further pertains to the amplification of single viral genomes are amplified and confined within the droplets, followed by extraction and isolation of the single-droplet contents for sequencing analysis.

### CROSS-REFERENCE TO RELATED APPLICATION

The present application claims the benefit of U.S. Provisional Application Ser. No. 63/488,802, filed Mar. 7, 2023, which is hereby incorporated by reference herein in its entirety, including any figures, tables, or drawings.

### REFERENCE TO SEQUENCE LISTING

The Sequence Listing for this application is labeled “UHK319X.xml” which was created on Jan. 30, 2024 and is 26,590 bytes. The entire content of the sequence listing is incorporated herein by reference in its entirety.

### BACKGROUND OF THE INVENTION

With as many as 107 viruses per milliliter of seawater and 109 per gram of marine sediment and soil in terrestrial environments, viruses are the most abundant and genetically diverse entities in the ecosystem. Current virus analytical techniques, including gene sequencing, infection testing, and immune response detection, measure only the average response from a highly heterogeneous population of viruses, thereby losing important information on a small but potentially relevant subpopulation. Although some single-cell manipulation and analysis techniques have been developed, they fail to work for a single virus due to its small size. The manipulation resolution of conventional methods, such as micromanipulation and laser capture microdissection, are beyond the scale of a virus (normally smaller than 100 nm). On the other hand, current detection tools, such as fluorescence detection and impedance detection, are unable to recognize the weak signal of a single virus.

Therefore, there remains a need for novel methods of isolating and analyzing viruses.

### BRIEF SUMMARY OF THE INVENTION

The subject invention pertains to methods of isolating, amplifying, and extracting single viral genomes from a sample using a microfluidic pipeline. After encapsulating a single virus into a droplet, the genome of the virus can be amplified. In certain embodiments, an integrated system is used to extract the single viral genomes from individual droplets and isolate them into arrays of microtubes. Through further fragmentation and amplification using specific barcodes, the processed genomes can be pooled and sequenced, providing a library of whole-genome sequences at single-virus level. In certain embodiments, the individual virus and subsequent amplified genomes remain separated from any other viruses or genomes thereof until the barcoded genomes are pooled for sequencing (i.e., no cross-contamination is induced during each extraction, and nearly whole genomes can be sequenced at single-virus level).

In certain embodiments, two immiscible flows can be used to encapsulate a single virus in a single droplet with uniform size distribution. Each droplet can be moved to a distinct compartment. The separated compartments serve as ideal vessels to isolate and manipulate individual viruses with several unique advantages. In certain embodiments, there are no restrictions on the target size or signal intensity; single viruses can be simply encapsulated within the droplets. In certain embodiments, the movement of each droplet is separated by the carrier oil; single viruses can be processed within the droplets without any cross-contamination. In certain embodiments, the droplets can be generated and manipulated with high throughput, allowing processing and analysis of a large number of viruses.

The subject methods can be used to deduce full-genome information at single-virion level. This allows detecting the viral sequence diversity in a mixed virus population at single-virion level in a high-throughput manner, which was previously not feasible. This approach can help to, for example, map key genetic information (e.g., antiviral resistance mutations) at a single-virion level, which can be useful for detecting important variants with an extremely low prevalence in a population (e.g., a virion with multiple antiviral resistance and/or antigenic mutations).

### BRIEF DESCRIPTION OF THE DRAWINGS

FIG. 1. Schematic showing the core concept of single-virus analysis. Microfluidic droplets are used to separate and amplify single viral genomes. Through extraction of individual droplet into each tube, the viruses can be analyzed at single-virus level.

FIG. 2. Specific pipeline of the single-virus analysis. Individual viruses are encapsulated into the droplets, with their genomes amplified to generate detectable signals. Subsequently, an automatic system is used to extract the single viral genomes from individual droplets and isolate them into arrays of microtubes. Through further fragmentation and amplification using specific barcodes, the processed genomes are pooled and sequenced, providing a library of single-virus genotypes at whole-genome level.

FIG. 3 Micrograph of the droplets after RT-PCR. The droplets are uniform in size, and the positive ones generate higher green fluorescence signals than the negative ones.

FIG. 4 Schematic showing the setup of the fluorescence detection system. The beam excited by a laser source is aligned and focused on the microfluidic channel. The emitted fluorescence signal is transferred into electrical signals by a PMT to identify the arrival of positive droplets.

FIG. 5 Design of the microfluidic channel. Processed droplets are reinjected and spaced in the main channel, followed by extraction in the square chamber.

FIGS. 6A-6B (FIG. 6A) Micrograph showing the balance of two flows without interference in their respective channel. (FIG. 6B) The matching of flow rates for balance of two flows.

FIGS. 7A-7B. Micrographs showing extraction of single-droplet contents. While positive droplets are extracted into upper channel (FIG. 7A), negative droplets flow with carrier oil into the lower channel (FIG. 7B).

FIG. 8. Diagrams showing the sequencing results of five extracted single-virus samples. Nearly whole genomes can be sequenced.

FIG. 9. Gel electrophoresis image for carry-over test. 5 positive and 5 negative samples are alternately collected and tested, and 5 negative are first included as a control.

FIG. 10. Diagram showing the CT values of 200 collected samples. The x-axis denotes the CT value tested for H1 genomes, while the y-axis shows the CT value tested for H3 genomes.

### BRIEF DESCRIPTION OF THE SEQUENCES

SEQ ID NO: 1: forward primer of the NS segment of the influenza virus
SEQ ID NO: 2: reverse primer of the NS segment of the influenza virus
SEQ ID NO: 3: Primer PB2-1F
SEQ ID NO: 4: Primer PB2-1250R
SEQ ID NO: 5: Primer PB2-1105F
SEQ ID NO: 6: Primer PB2-2341R
SEQ ID NO: 7: Primer PB1-1F
SEQ ID NO: 8: Primer PB1-1243R
SEQ ID NO: 9: Primer PB1-1124F
SEQ ID NO: 10: Primer PB1-2341R
SEQ ID NO: 11: Primer PA-1F
SEQ ID NO: 12: Primer PA-1256R
SEQ ID NO: 13: Primer PA-974F
SEQ ID NO: 14: Primer PA-2233R
SEQ ID NO: 15: Primer HA-1F
SEQ ID NO: 16: Primer HA-1079R
SEQ ID NO: 17: Primer HA-753F
SEQ ID NO: 18: Primer NP-1F
SEQ ID NO: 19: Primer NP-949R
SEQ ID NO: 20: Primer NP-647F
SEQ ID NO: 21: Primer NP-1565R
SEQ ID NO: 22: Primer NA-1F
SEQ ID NO: 23: Primer NA-856R
SEQ ID NO: 24: Primer NA-540F
SEQ ID NO: 25: Primer NA-1413R
SEQ ID NO: 26: Primer M-1F
SEQ ID NO: 27: Primer M-1027R
SEQ ID NO: 28: Primer NS-1F
SEQ ID NO: 29: Primer NS-890R

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “genome”, “genomic”, “genetic material” or other grammatical variation thereof as used herein refers to genetic material from any organism or virus. Genetic material can be viral genomic DNA or RNA, nuclear genetic material, such as genomic DNA or genomic RNA. It can also represent the genetic material coming from a natural or artificial mixture or a mixture of genetic material from several organisms or viruses.

As used herein, the phrase “hybridizes with” when used with respect to two sequences indicates that the two sequences are sufficiently complementary to each other to allow nucleotide base pairing between the two sequences. Sequences that hybridize with teach other can be perfectly complementary but can also have mismatches to a certain extent. Depending upon the stringency of hybridization, a mismatch of up to about 5% to 20% between the two complementary sequences would allow for hybridization between the two sequences. Typically, high stringency conditions have higher temperature and lower salt concentration and low stringency conditions have lower temperature and higher salt concentration. High stringency conditions for hybridization are preferred.

Also, two sequences that correspond to each other, for example, a primer binding sequence and a primer sequence or a sequencing primer binding sequence and a sequencing primer sequence, have at least 90% sequence identity, preferably, at least 95% sequence identity, even more preferably, at least 97% sequence identify, and most preferably, at least 99% sequence identity, over at least 70%, preferably, at least 80%, even more preferably, at least 90%, and most preferably, at least 95% of the sequences. Alternatively, two sequences that correspond to each other are reverse complementary to each other and have at least 90% perfect matches, preferably, at least 95% perfect matches, even more preferably, at least 97% perfect matches, and most preferably, at least 99% perfect matches in the reverse complementary sequences, over at least 70%, preferably, at least 80%, even more preferably, at least 90%, and most preferably, at least 95% of the sequences. Thus, two sequences that correspond to each other can hybridize with each other or hybridize with a common reference sequence over at least 70%, preferably, at least 80%, even more preferably, at least 90%, and most preferably, at least 95% of the sequences. Preferably, two sequences that correspond to each other are 100% identical over the entire length of the two sequences or 100% reverse complementary over the entire length of the two sequences.

As used herein, the term “next-generation sequencing” or “sequencing” refers to the so-called parallelized sequencing-by-synthesis or sequencing-by-ligation platforms, for example, those currently employed by, for example, Illumina, Life Technologies, and Roche. Next-generation sequencing methods may also include Nanopore sequencing methods or electronic-detection based methods such as Ion Torrent technology commercialized by Life Technologies. Illumina technology may be used in the methods disclosed herein to sequence the genome libraries. In certain embodiments, the genomic regions are processed to sequence genomic regions as described, for example, in Illumina NovaSeq 6000 Sequencing System Guide (Oct. 12, 2020). The content of the brochure is herein incorporated by reference in its entirety.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

The term “biological sample” or “sample from a subject” encompasses a variety of sample types obtained from an organism, particularly sample types that contain at least one virus. The term encompasses bodily fluids such as blood, blood components, saliva, nasal mucous, serum, plasma, cerebrospinal fluid (CSF), urine and other liquid samples of biological origin, solid tissue biopsy, tissue cultures, or supernatant taken from cultured patient cells. In the context of the present disclosure, the biological sample is typically a bodily fluid with detectable amounts of virus. The biological sample can be processed prior to assay, e.g., to remove cells or cellular debris. The term encompasses samples that have been manipulated after their procurement, such as by treatment with reagents, solubilization, sedimentation, or enrichment for certain components.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Isolating Single Viruses

This disclosure provides materials and methods that solve the problems associated with conventional methods for sequencing viral genomes. In certain embodiments, through the adoption of two immiscible flows, a buffer or water (e.g., PBS solution, deionized water) and a fluorinated oil (e.g., HFE 7500 oil, FC-40), droplet microfluidics enables generating pico-litered droplets with uniform size distribution. In certain embodiments, the sample containing the viruses can be diluted to a concentration of less than about 106 PFU/mL, 105 PFU/mL, 10⁴ PFU/mL, 103 PFU/mL, or 102 PFU/mL. In preferred embodiments, at low concentrations below 10⁴ PFU/mL, single viruses can be suspended in the PBS solution and encapsulated into the droplets. As a result, each virus can be independently processed and analyzed within the droplets and separated from other virus candidates. The viruses can be from a biological sample or from the environment. Examples of environments include recreational water, surfaces, and beach sand. Recreational water is any water in which recreation occurs and includes recreational bodies of water such as swimming pools, ponds, lakes, rivers, and oceans. Surfaces can include hospitals, schools, and food processing facilities.

Samples can be analyzed with a fluorogenic or colorimetric substrate in the aqueous phase of the droplets to determine the sequence of a target virus.

In certain embodiments, each droplet containing a single virus can be separated into single compartments to serve as vessels to isolate and manipulate individual viruses. Droplet extraction is performed in a rectangular chamber, in which a PBS stream flows side-by-side with the droplet emulsions between two electrodes. When one positive droplet is detected, a pulse signal is triggered and output to the electrode, activating an electric field to recover the contained single viral genomes into the aqueous stream, whereas the negative droplets flow along with the carrier oil into the lower channel in the absence of electric field. Subsequently, the dispensing setup controlled by an electro-pneumatic valve is actuated to start an aqueous flow, rapidly ejecting them into an aligned microtube. Upon the samples accommodated, the collection platform is rotated to align another tube with the nozzle for next cycle of extraction.

In certain embodiments the volume of the liquid of a single droplet is about 50 pL, corresponding to the diameter of about 45 μm. In some cases, the total volume of aqueous phase components, including but not limited to clinical sample or single virus from a clinical sample, that is used to generate droplets is from about 0.01 pL to about 100 pL or from about 0.1 pL to about 10 L.

In some cases, the total number of droplets of a plurality of droplets (e.g., containing a single virus) is at least about 400,000. In other embodiments, up to a million droplets are formed. In some cases, the total number of droplets of a plurality of droplets (e.g., containing a virus) is from about 3,000 to about 5,000.

### Virus RNA Detection

Any detection method or system able to detect a labelled nucleotide can be used in methods according to embodiments of the present invention and such appropriate detection methods and systems are well-known in the art. In certain embodiments, fluorescent microscopes, fluorescence scanners, spectrofluorometers and microplate readers, flow cytometers, or real-time PCR machine can be used to detect fluorescence.

In certain embodiments, the detection of the at least one single-stranded or double stranded nucleic acid is carried out in an enzyme-based nucleic acid amplification method. In preferred embodiments, fluorescent probes and primers are encapsulated into the droplets to amplify the viral genomes.

The expression “enzyme-based nucleic acid amplification method” relates to any method wherein enzyme-catalyzed nucleic acid synthesis occurs.

Such an enzyme-based nucleic acid amplification method can be preferentially selected from the group constituted of LCR, Q-beta replication, NASBA, LLA (Linked Linear Amplification), TMA, 3SR, Polymerase Chain Reaction (PCR), notably encompassing all PCR based methods known in the art, such as reverse transcriptase PCR (RT-PCR), simplex and multiplex PCR, real time PCR, end-point PCR, quantitative or qualitative PCR and combinations thereof. These enzyme-based nucleic acid amplification method are well known to the man skilled in the art and are notably described in Saiki et al. (1988) Science 239:487, EP 200 362 and EP 201 184 (PCR); Fahy et al. (1991) PCR Meth. Appl. 1:25-33 (3SR, Self-Sustained Sequence Replication); EP 329 822 (NASBA, Nucleic Acid Sequence-Based Amplification); U.S. Pat. No. 5,399,491 (TMA, Transcription Mediated Amplification), Walker et al. (1992) Proc. Natl. Acad. Sci. USA 89:392-396 (SDA, Strand Displacement Amplification); EP 0 320 308 (LCR, Ligase Chain Reaction); Bustin & Mueller (2005) Clin. Sci. (London) 109:365-379 (real-time Reverse-Transcription PCR).

In some embodiments, the enzyme-based nucleic acid amplification method is selected from the group consisting of Polymerase Chain Reaction (PCR) and Reverse-Transcriptase-PCR (RT-PCR), and real time PCR or RT-PCR. In other embodiments, the enzyme-based nucleic acid amplification method is a real time, optionally multiplex, PCR, quantitative PCR or RT-PCR method.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step. The polymerase reactions are incubated under conditions in which the primers hybridize to the target sequences and are extended by a polymerase. The amplification reaction cycle conditions are selected so that the primers hybridize specifically to the target sequence and are extended.

Successful PCR amplification requires high yield, high selectivity, and a controlled reaction rate at each step. Yield, selectivity, and reaction rate generally depend on the temperature, and optimal temperatures depend on the composition and length of the polynucleotide, enzymes and other components in the reaction system. In addition, different temperatures may be optimal for different steps. Optimal reaction conditions may vary, depending on the target sequence and the composition of the primer. Thermal cyclers such as, for example, real-time PCR systems provide the necessary control of reaction conditions to optimize the PCR process for a particular assay. For instance, a real-time PCR system may be programmed by selecting temperatures to be maintained, time durations for each cycle, number of cycles, and the like. In some embodiments, temperature gradients may be programmed so that different sample wells may be maintained at different temperatures, and so on.

In certain embodiments, the target nucleic acid sequence can be RNA or DNA. RNA or DNA can be artificially synthesized or isolated from natural sources. In some embodiments, the RNA target nucleic acid sequence can be a ribonucleic acid such as RNA, mRNA, piRNA, tRNA, rRNA, ncRNA, gRNA, shRNA, siRNA, snRNA, miRNA and snoRNA More preferably the DNA or RNA is biologically active or encodes a biologically active polypeptide. The DNA or RNA template can also be present in any useful amount.

Reverse transcriptases useful in the present invention can be any polymerase that exhibits reverse transcriptase activity. Preferred enzymes include those that exhibit reduced RNase H activity. Several reverse transcriptases are known in the art and are commercially available (e.g., from Bio-Rad Laboratories, Inc., Hercules, CA; Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). In some embodiments, the reverse transcriptase can be Avian Myeloblastosis Virus reverse transcriptase (AMV-RT), Moloney Murine Leukemia Virus reverse transcriptase (M-MLV-RT), Human Immunovirus reverse transcriptase (HIV-RT), EIAV-RT, RAV2-RT, C. hydrogenoformans DNA Polymerase, rTth DNA polymerase, SUPERSCRIPT I, SUPERSCRIPT II, and mutants, variants and derivatives thereof. It is to be understood that a variety of reverse transcriptases can be used in the present invention, including reverse transcriptases not specifically disclosed above, without departing from the scope or preferred embodiments disclosed herein.

DNA polymerases useful in the present invention can be any polymerase capable of replicating a DNA molecule. Preferred DNA polymerases are thermostable polymerases and polymerases that have exonuclease activity, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus woesei (Pwo) and other species of the Pyrococcus genus, Bacillus sterothemophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof.

Many DNA polymerases are known in the art and are commercially available (e.g., from Bio-Rad Laboratories, Inc., Hercules, CA; Boehringer Mannheim Corp., Indianapolis, Ind.; Life Technologies, Inc., Rockville, Md; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). In some embodiments, the DNA polymerase can be Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, and active mutants, variants and derivatives thereof. It is to be understood that a variety of DNA polymerases can be used in the present invention, including DNA polymerases not specifically disclosed above, without departing from the scope or preferred embodiments thereof.

The reverse transcriptase can be present in any appropriate ratio to the DNA polymerase. In some embodiments, the ratio of reverse transcriptase to DNA polymerase in unit activity is greater than or equal to 3. One of skill in the art will appreciate that other reverse transcriptase to DNA polymerase ratios are useful in the present invention.

In a preferred embodiment, the reactions according to the invention can also contain further reagents suitable for a PCR step.

Such reagents are known to those skilled in the art, and include water, like nuclease-free water, RNase free water, DNAse-free water, PCR-grade water; salts, like magnesium, magnesium chloride, potassium; buffers such as Tris; enzymes; nucleotides like deoxynucleotides, dideoxunucleotides, dNTPs, dATP, dTTP, dCTP, dGTP, dUTP and modified nucleotides such as deaza-, locked nucleic acid, and peptide nucleic acid; other reagents, like DTT and/or RNase inhibitors; and polynucleotides like polyT and polydT.

In certain embodiments, the droplets can contain one or more detection reagents. The detection reagents can be genotype specific nucleic acid detection reagents, proteins (e.g., antibodies, lectins, fibrinogen) and other molecules. Exemplary genotype specific nucleic acid detection reagents include, for example, nucleic acid probes. Genotype specific detection reagents may be specific for a particular virus. Exemplary nucleic acid probes include, but are not limited to, double-stranded probes, such as the double-stranded probes described in U.S. Pat. Nos. 5,928,862; or 9,194,007; Molecular Beacon probes, such as those described in WO 98/10096; TaqMan probes, such as those described in U.S. Pat. Nos. 5,210,015; 5,487,972; 5,538,848; 5,723,591; and 6,258,569; scorpion probes; light cycler probes; LUX probes; and amplifluor probes. Such probes can be used for detection by, e.g., adding the droplets containing the probes under conditions enabling hybridization to the target virus, then lysing the target viruses by heating at, for example, about 50° C. for about 1 h, optionally amplifying a target nucleotide sequence, and detecting the presence or absence of a nucleic acid sequence with the nucleic acid probe. In some embodiments, labeled antibodies or other molecules specific to particular viruses are used. Such labeled molecules can be specific also to a virus, allowing for identification of the virus.

In preferred embodiments, a FAM-labeled TaqMan probe is designed for fluorescence detection of the viral genome, such as, for example, the NS segment of the influenza virus. In certain embodiments, a least 1, 2, 3, 4 5, 6, 7, 8, 9, 10, 11, 12, 13, 14, 15, 16, 17, 18, 19, 20 pairs of oligonucleotide primers can be added to a droplet containing a virus. In certain embodiments, each pair is specific for a virus, particularly a gene of the virus. In preferred embodiments, at least two pairs of primers are added to the droplet in which one pair amplifies a region of the viral genome and a second pair amplifies the remaining viral genome or a substantial portion of the remaining genome, such as, for example, at least 60% of the genome is amplified. In preferred embodiments, the expected amplicon size does not exceed about 1300 bp; the amplicon can be about 500 bp to about 2000 bp. In preferred embodiments, each primer pair can amplify a portion of a gene encoded by the viral genome. The portion can be about 900 bp about 1500 bp.

### Targets of Nucleotide Detection

In certain embodiments, the methods provided by the subject invention can be used to isolate one or more virus and the genome thereof (also referred to as “target sequence(s)”, “target nucleic acid sequence(s)”, or “target nucleotide sequence(s)” herein).

In certain embodiments, the methods of the subject invention can be used to detect and sequence RNA viruses, such as, for example, Middle East respiratory syndrome-related coronavirus (MERS), severe acute respiratory syndrome coronavirus (SARS-CoV); SARS-CoV-2, including genetic variants, such as, for example, B.1.351 (South African), B.1.17 (United Kingdom), P.1 (Brazil), B.1.429 (California), and B.1.617 (India) when compared to the wild-type Wuhan virus; influenza, including the 4 types of influenza (A, B, C, and D) and genetic variants of the genes encoding hemagglutinin (HA) and neuraminidase (NA); enteroviruses, including genetic variants of genes that can be used to differentiate between Enteroviruses A-L and serotypes of Enteroviruses A-L; human immunodeficiency virus (HIV), including genetic variants of genes that can be used to differentiate between the two groups (HIV-1 and HIV-2) and the various subtypes (clades), which can be differentiated by, for example, the envelop-encoding region; DNA viruses, including human papillomavirus (HPV), including genetic variants of genes, such as, E1, E2, E3, E4, E5, E6, E7, L1, L2, and the Long Coding Region (LRC) of HPV that can be used differentiate common strains, such as, for example, HPV 16, 18, 31, 33, and 45, and other strains described in Muñoz N, Bosch F X, de Sanjosé S, Herrero R, Castellsagué X, Shah K V, Snijders P J, Meijer C J; International Agency for Research on Cancer Multicenter Cervical Cancer Study Group. Epidemiologic classification of human papillomavirus types associated with cervical cancer. N Engl J Med. 2003 Feb. 6; 348(6):518-27. doi: 10.1056/NEJMoa021641, which is hereby incorporated by reference.

### Methods of Isolating and Amplifying Viral Genomes

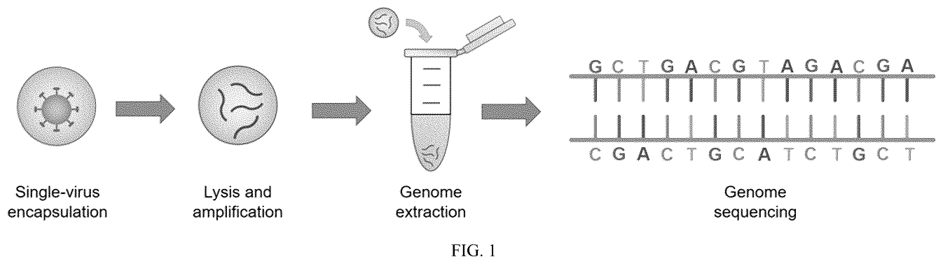

In certain embodiments, the methods can be used for rapidly analyzing the genome of an individual virus in a sample by partitioning the sample into a plurality of droplets and amplifying the viral genome in droplet. The concept of this method is using droplets to isolate and process single viruses, followed by extraction of single viral genomes for sequencing analysis (FIG. 1). Specifically, single viruses are first encapsulated into microfluidic droplets in a flow-focusing channel. Then through droplet-based RT-PCR, the whole genomes of single viruses are amplified and confined within the droplets. The thermal cycling is set as 50° C. for 60 min for reverse transcription, 95° C. for 10 min for reverse transcriptase inactivation, 25 cycles of genomic amplification at 95° C. for 30 s and at 60° C. for 2 min, and a final amplicon extension at 98° C. for 10 min. Subsequently, the droplets are processed by an automatic system to extract single-droplet contents. Finally, after further PCR for amplification, the single-virus samples can be sequenced at full-genome level.

In certain embodiments, a droplet generator operates by microchannel flow focusing to generate droplets. The aqueous and oil flow rates are set at 200 μL/h and 600 μL/h, resulting the droplet diameter of about 45 μm. The resulting droplets can have any suitable shape and size. The droplets can be spherical, when shape is not constrained. The number of droplets generated can depend upon a variety of factors including, but not limited to, the instrumentation utilized and manner of droplet generation (e.g., bulk agitation or serial generation), the droplet chemistry, droplet volume, the volume of aqueous and/or non-aqueous phases consumed during droplet generation, the volume of the sample analyzed, and the number of target microorganisms to be partitioned.

In certain embodiments, each droplet can be formed, can encapsulate a virus, and then be collected in a reservoir, such as vial, a test tube, a well of a plate, a chamber, or compartment. In an alternative embodiment, droplets are formed and enter a microfluidic, flow-focusing channel. In some embodiments, the droplets can be collected in a compartment suitable for nucleotide amplification, including, for example, a PCR tube or microplate. In such an embodiment, a single-file line of droplets forms in the channel.

In certain embodiments, detection can be detection of fluorescence or absorbance of a fluorophore or chromophore. For example, a digestion of a fluorogenic or colorimetric substrate can be detected by detecting droplets that exhibit characteristic absorbance or fluorescence. In certain embodiments, fluorogenic probes or intercalating dyes can be used to respectively detect the presence or absence of a nucleic acid sequence or double-stranded nucleic acid in general.

In certain embodiments, droplets are identified as positive or negative. Positive droplets are those that contain a virus and negative droplets are those that do not contain a virus. Analysis can be performed manually, e.g., using an optical microscope or in an automated fashion. In some cases, the automated detection is performed by serially flowing the droplets through a detection region configured to detect absorbance, transmission or emission (e.g., fluorescence) at one or more wavelengths. In some cases, a spacing fluid (e.g., oil) is added to droplets flowing in a channel to a detection region that is operatively disposed with respect to a detector and/or an excitation light source. In some cases, the automated detection is performed by a high throughput particle counting system in a volume of droplets.

In certain embodiments, an optical setup is used to detect and identify the positive droplets. A laser source with a wavelength of, for example, 488 nm is used to excite the fluorescence signal for the, for example, FAM probe. The wavelength of the laser source is specific to the probe. Through four lenses, the laser beam is reflected to the plan fluorite objective and focused to the microfluidic channel. The excited fluorescence signal then propagates backward and is separated into two beams through a splitter, with 90% of light received by a photo-multiplier tube (PMT) for fluorescence detection, and the remaining 10% for high-speed real-time imaging. The triggered signal will be processed by a digital acquisition board coupled with a, for example, LabVIEW program.

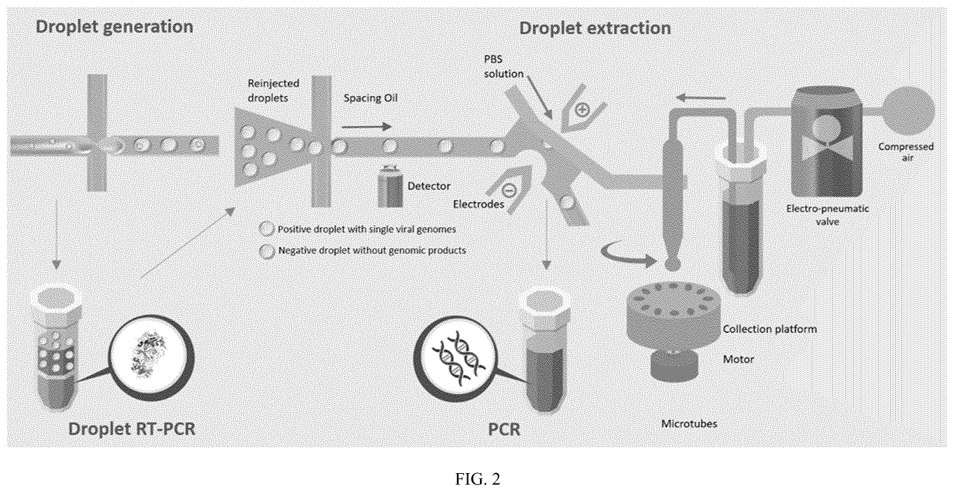

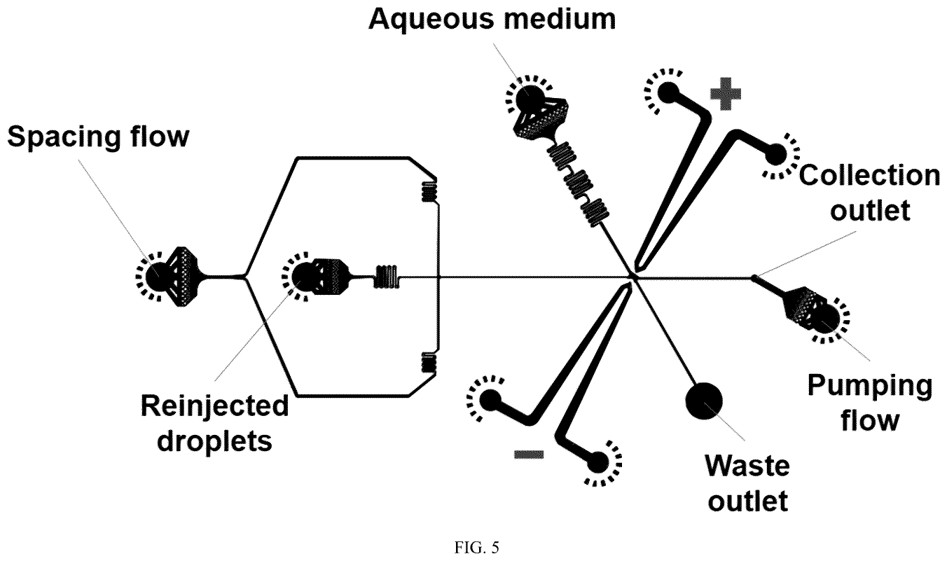

In certain embodiments, after the positive droplets with the amplified viral genomes have been identified, the encapsulated viral genomes are extracted in a microfluidic channel, as shown in FIG. 5. The droplets are reinjected and spaced by an oil flow in a cross junction as shown in FIG. 2. In certain embodiments, then droplets are guided into a square chamber to extract single droplet contents. Two streams of aqueous medium and droplet emulsions flow in the chamber. The channel has a height of about 45 μm, which is fabricated using a typical soft lithography replica molding technique. However, it is not limited to polydimethylsiloxane (PDMS)-based device; other materials, such as polymethyl methacrylate (PMMA) can also be used. In certain embodiments, upon detection of one positive droplet in the square chamber, a pulse signal output by, for example, the LabVIEW program and amplified by a high-voltage amplifier, is applied to electrodes on either side of the square chamber, extracting the contained single viral genomes into the aqueous medium in the upper channel. By contrast, the negative droplets flow along with the carrier oil into the lower channel in absence of the electric field. In certain embodiments, a wait time of about 30 to about 100 ms is performed before the air dispensing. The dispensing setup comprises an electro-pneumatic valve, a glass tube, and a connection tubing. With one end connected to compressed air, this valve can output different pressures at the other end according to different DC voltage supplies. Thus, the ejecting flow of outer phase can be driven with an appropriate voltage to rapidly eject droplets into the microtube. At the voltage supply of about 5 to about 10 V, the valve can drive an aqueous flow from the tube to the channel by air pressure. About 5 to about 20 μL of aqueous solution containing the single-virus genomes can be rapidly ejected into the microtube in 150 ms. The rotation platform is controlled by a step motor. A step motor was fixed on the bottom to control its rotation, which was actuated by a transistor-transistor logic (TTL) signal to rotate at the step angle of about 0.9°. When 20 cycles of pulse signals were applied, the step motor actuated the platform to rotate 18° in 20 ms, aligning another tube with the nozzle. At this setup, the whole cycle of extraction, dispensing, and collection can be completed in 200 ms, resulting in the highest extraction frequency of 5 Hz.

To achieve a faithful recovery, the two streams must be balanced without interference in their respective channels. Otherwise, the extracted droplet contents tend to be lost when the aqueous flow enters the lower channel; or the samples may be contaminated to hamper subsequent sequencing when the oil flow enters the upper channel. The balanced interface is obtained by accurate regulation of the two flow rate at a ratio of about 1:3, such as, for example, 1000 μL/h and 3200 μL/h, respectively.

After microfluidic processing, the acquired single viral genomes can be amplified with one round of PCR within the tube to increase the genome concentration to a detectable level. Random primers are used to cut the full-length genomes into small segments that are within the sequencing read length. Then another round of PCR is conducted to label the samples for pooled sequencing. Unique barcodes are added to tag the fragmented genomes in each sample. In certain embodiments, next-generation sequencing (NGS) is conducted to probe the genetic diversity at single-virus level.

### Kits

In certain embodiments, kits for the isolation and amplification of one or more viral genomes are provided. The kits can include reagents for forming droplets to encapsulate a virus. The kit can include instructions for carrying out the methods described herein.

### Materials and Methods

The purified virus was diluted to 1:10000 and added into the digital droplet PCR mix (1-Step RT-ddPCR Advanced Kit for Probes, Bio-Rad) with the ratio according to the manufacturer's suggestion as the aqueous phase. Fluorinated oil (HFE7500, 3M) with a surfactant (008, RAN Biotechnologies) concentration of 5% (w/w) was used as the oil phase. Microfluidic droplets were generated by adjusting the flow rates of the aqueous and oil phases at 200 μL/h and 600 μL/h, respectively. The oil phase in the generated emulsions was then replaced by another fluorinated oil (FC-40, Sigma-Aldrich) with 5% surfactant. The droplets are subjected to the recommended RT-PCR thermal cycles: 50° C. for 60 min for reverse transcription, 95° C. for 10 min for reverse transcriptase inactivation, 40 cycles of genomic amplification at 95° C. for 30 s and at 60° C. for 2 min, and a final amplicon extension at 98° C. for 10 min.

The resultant PCR products were run by an agarose gel, where 100-500 base pairs of each sample was cut and purified (QIAquick Gel Extraction Kit, Qiagen). Samples linked with different barcodes were pooled into one sample at the same concentration for Illumina NovaSeq 6000 next-generation sequencing services provided by HKU CPOS Genomics Core.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Encapsulating Viruses

The concept of this method is using droplets to isolate and process single viruses, followed by extraction of single viral genomes for sequencing analysis (FIG. 1). Specifically, single viruses are first encapsulated into microfluidic droplets in a flow-focusing channel. Then through droplet-based RT-PCR, the whole genomes of single viruses are amplified and confined within the droplets. Subsequently, the droplets are processed by an automatic system to extract single-droplet contents. Finally, after further PCR for amplification, the single-virus samples can be sequenced at full-genome level (FIG. 2).

### Example 2—Viral Genome Amplification

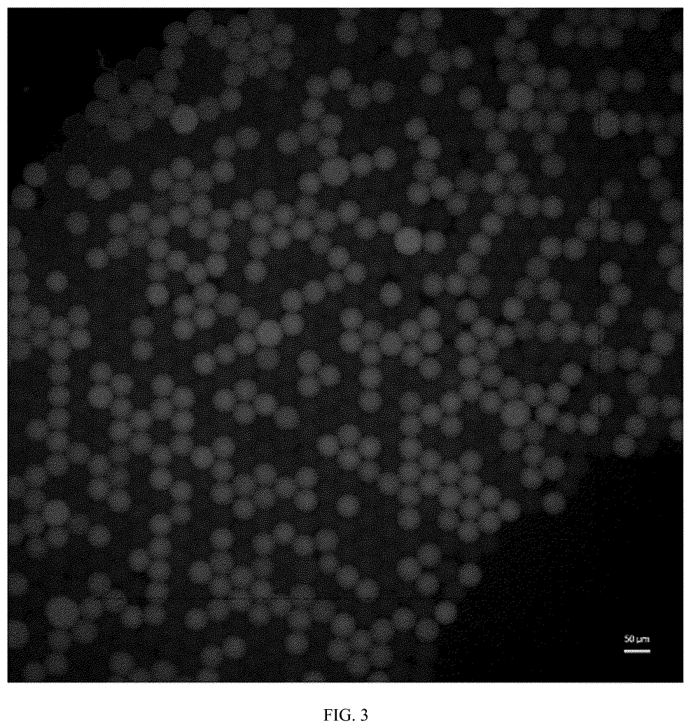

Fluorescent probes and primers are encapsulated into the droplets to amplify the viral genomes. A FAM-labeled TaqMan probe is designed for fluorescence detection of the viral genomes. The probe targets the NS segment of the influenza virus at around 500 bp, with forward primer 5′-GAAGAGGGAGCAATTGTTGGCG-3′ (SEQ ID NO: 1) and reverse primer 5′-CAGAGACTCGAACTGTGTTATCATTCC-3′ (SEQ ID NO: 2). For segments except NS, gene-specific primers were used for targeted flanking. Extra pairs of primer that anneal on the conserved middle part of the segments were used for sufficient amplification of segments except NS, as shown in Table 1. Consequently, the expected amplicon size does not exceed 1300 bp; the amplification bias between different lengths of segments can be eliminated. For NS segment, universal flu primers, Uni12 and Uni13, with high binding efficiencies were used for amplification. In addition, the primer that binds on the 3′ end of HA segment was Uni13. The thermal cycling is set as 50° C. for 60 min for reverse transcription, 95° C. for 10 min for reverse transcriptase inactivation, 25 cycles of genomic amplification at 95° C. for 30 s and at 60° C. for 2 min, and a final amplicon extension at 98° C. for 10 min. After RT-PCR, the positive droplet encapsulating single viruses can emit green fluorescence signals, while the negative ones show no signals, as shown in FIG. 3.

TABLE 1

| Primer | Sequence (5′ to 3′) |
|---|---|
| PB2-1F | TATTGGTCTCAGGGAGCGAAAGCAGGTC (SEQ ID NO: 3) |
| PB2-1250R | TCCTCTTGTGAAAATACCAT (SEQ ID NO: 4) |
| PB2-1105F | TATGAAGAGTTCACAATGGT (SEQ ID NO: 5) |
| PB2-2341R | ATATGGTCTCGTATTAGTAGAAACAAGGTCGTTT (SEQ ID NO: 6) |
| PB1-1F | TATTCGTCTCAGGGAGCGAAAGCAGGCA (SEQ ID NO: 7) |
| PB1-1243R | TTGAACATGCCCATCATCAT ((SEQ ID NO: 8) |
| PB1-1124F | AAATACCTGCAGAAATGCT (SEQ ID NO: 9) |
| PB1-2341R | ATATCGTCTCGTATTAGTAGAAACAAGGCATTT (SEQ ID NO: 10) |
| PA-1F | TATTCGTCTCAGGGAGCGAAAGCAGGTAC (SEQ ID NO: 11) |
| PA-1256R | AACTCATTCTGAATCCAACTTGC (SEQ ID NO: 12) |
| PA-974F | GGAAGGAACCCAATGTTGTTAAA (SEQ ID NO: 13) |
| PA-2233R | ATATCGTCTCGTATTAGTAGAAACAAGGTACTT (SEQ ID NO: 14) |
| HA-1F | TATTCGTCTCAGGGAGCAAAAGCAGGGG (SEQ ID NO: 15) |
| HA-1079R | AATGGCTCCAAATAGACCTCTG (SEQ ID NO: 16) |
| HA-753F | GGGAGGATGAACTATTACTGGAC (SEQ ID NO: 17) |
| NP-1F | TATTCGTCTCAGGGAGCAAAAGCAGGGTA (SEQ ID NO: 18) |
| NP-949R | CTATTCCGACTAGAGAGTATCCC (SEQ ID NO: 19) |
| NP-647F | TCAATGATCGGAACTTCTGGAG (SEQ ID NO: 20) |
| NP-1565R | ATATCGTCTCGTATTAGTAGAAACAAGGGTATTTTT (SEQ ID NO: |
| | 21) |
| NA-1F | TATTGGTCTCAGGGAGCAAAAGCAGGAGT (SEQ ID NO: 22) |
| NA-856R | TCTCTGCACACACACATCAC (SEQ ID NO: 23) |
| NA-540F | GGCTGGCTAACAATCGGAAT (SEQ ID NO: 24) |
| NA-1413R | ATATGGTCTCGTATTAGTAGAAACAAGGAGTTTTTT (SEQ ID NO: |
| | 25) |
| M-1F | TATTCGTCTCAGGGAGCAAAAGCAGGTAG (SEQ ID NO: 26) |
| M-1027R | ATATCGTCTCGTATTAGTAGAAACAAGGTAGTTTTT (SEQ ID NO: |
| | 27) |
| NS-1F | TATTCGTCTCAGGGAGCAAAAGCAGGGTG (SEQ ID NO: 28) |
| NS-890R | ATATCGTCTCGTATTAGTAGAAACAAGGGTGTTTT (SEQ ID NO: |
| | 29) |

### Example 3—Identifying Droplets Containing a Virus

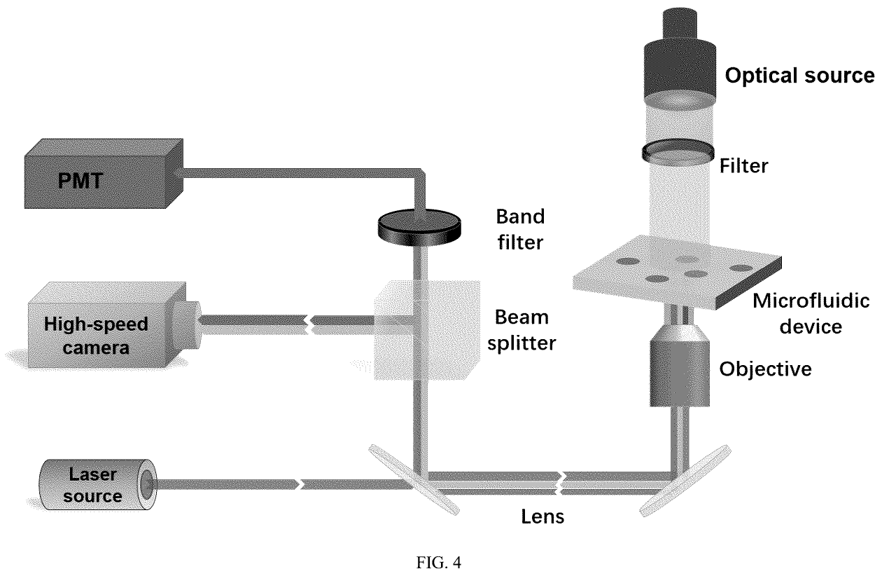

An optical setup is used to detect and identify the positive droplets (FIG. 4). A laser source with a wavelength of 488 nm is used to excite the fluorescence signal. Through a series of lenses, the beam is reflected to the plan fluorite objective and focused to the microfluidic channel. The excited fluorescence signal then propagates backward and is separated into two beams through a splitter, with 90% of light received by a photo-multiplier tube (PMT) for fluorescence detection, and the remaining 10% for high-speed real-time imaging. The triggered signal will be processed by a digital acquisition board coupled with a LabVIEW program.

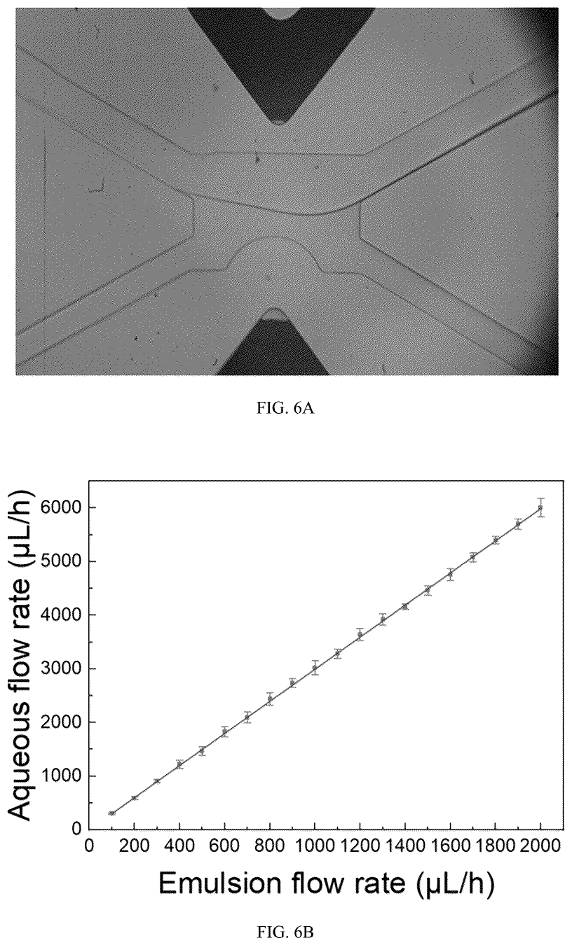

The encapsulated viral genomes are extracted in a microfluidic channel, as shown in FIG. 5. The droplets are reinjected and spaced in a cross junction. Then they are guided into a square chamber to extract single droplet contents. Two streams of aqueous medium and droplet emulsions flow in the chamber. To achieve a faithful recovery, the two streams must be balanced without interference in their respective channels. Otherwise, the extracted droplet contents tend to be lost when the aqueous flow enters the lower channel; or the samples may be contaminated to hamper subsequent sequencing when the oil flow enters the upper channel. The balanced interface is obtained by accurate regulation of the two flow rate at 1000 μL/h and 3200 μL/h, respectively (FIGS. 6A-6B).

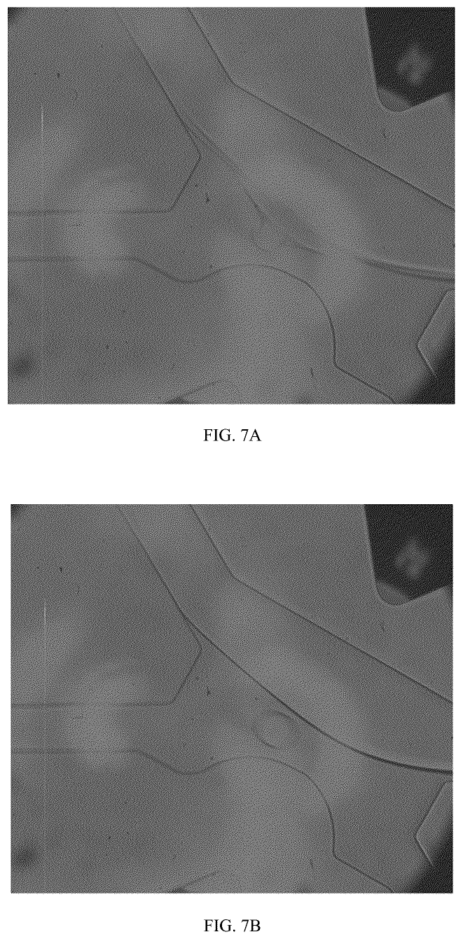

Upon detection of one positive droplet, a pulse signal output by the LabVIEW program and amplified by a high-voltage amplifier is applied to the electrodes, extracting the contained single viral genomes into the aqueous medium. By contrast, the negative droplets flow along with the carrier oil into the lower channel in absence of the electric field (FIGS. 7A-7B). To guarantee complete droplet extraction, a wait time of 30 ms was used before the start of the dispensing. The dispensing setup comprises an electro-pneumatic valve, a glass tube, and a connection tubing. At the voltage supply of 5 V, the valve can drive an aqueous flow from the tube to the channel by air pressuring. Around 5 μL of aqueous solution containing the single-virus genomes is rapidly ejected into the microtube in 150 ms. The rotation platform is controlled by a step motor. When 20 cycles of TTL signals were applied, the step motor actuated the platform to rotate 18° in 20 ms, aligning another tube with the nozzle. At this setup, the whole cycle of extraction, dispensing, and collection can be completed in 200 ms, resulting in the highest extraction frequency of 5 Hz.

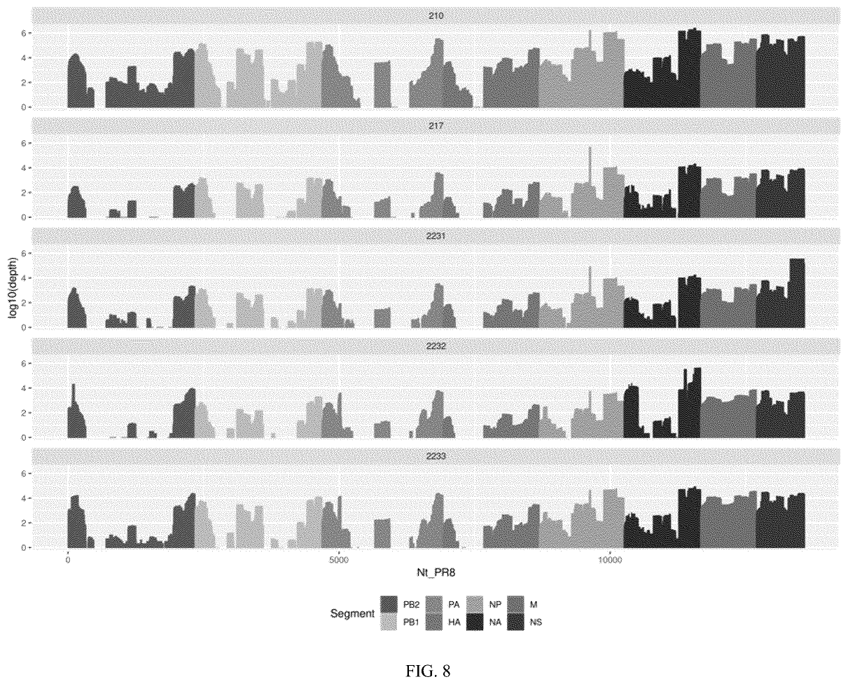

After microfluidic processing, the acquired single viral genomes are treated with one round of PCR to increase the genome concentration to a detectable level. Random primers are used to cut the full-length genomes into small segments that are within the sequencing read length. Then another round of PCR is conducted to label the samples for pooled sequencing. Unique barcodes are added to tag the fragmented genomes in each sample. Subsequently, next-generation sequencing (NGS) is conducted to probe the genetic diversity at single-virus level. Influenza virus, A/PR/8/34 (H1N1), with 8 separate segments of genomes is used as a model to test the efficiency of the single-virus analysis. As shown in FIG. 8, the NA, NP, M and NS segments of the 5 sequenced samples reached almost completed coverage; the PB1, PB2, PA, and HA segments reach most of the coverage.

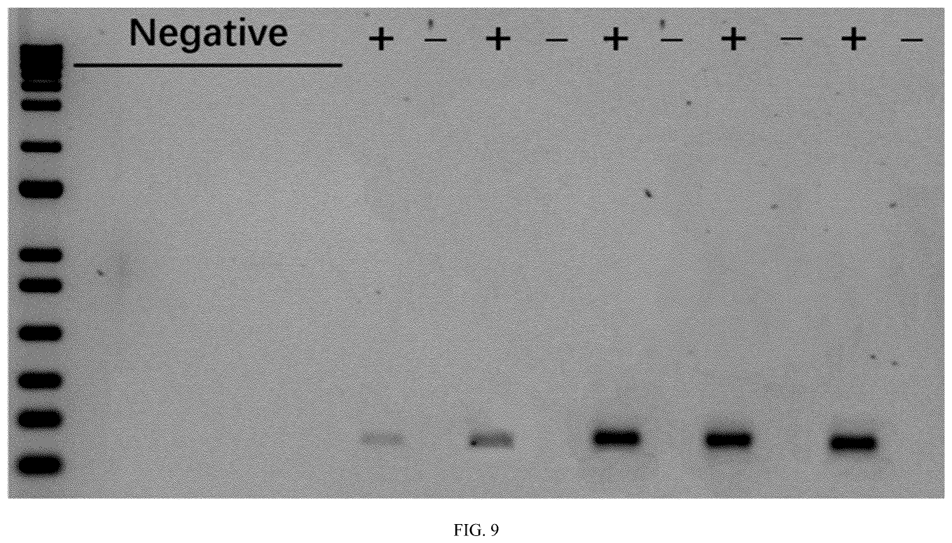

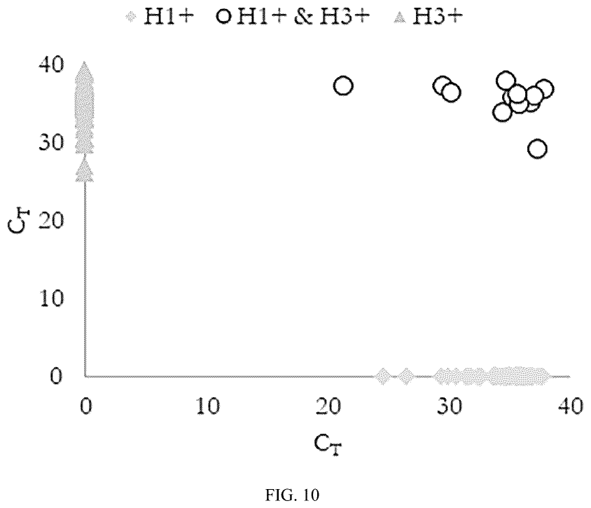

To verify no genome residual remained in each extraction to induce cross-contamination, we alternately extract 5 positive droplets and 5 negative droplets. Gel electrophoresis is performed to test the genome concentration in each sample. As shown in FIG. 9, the positive samples have distinct bands corresponding to the viral genomes, while the negative ones show no genomic signals, demonstrating no carry-over of viral genomes during the single-droplet extraction. Moreover, we test the integrity of the single-virus samples. Two different types of influenza viruses, H1 and H3, are encapsulated into the droplets. After extraction, specific primers and real-time PCR are used to test which genomes are included in each sample. In this test, each sample should associate entirely with either H1 or H3 genomes; only two-virus events would lead to the appearance of mixed profiles. As shown in FIG. 10, 94% of the 200 tested samples contain only one type of genomes, as expected.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Exemplary Embodiments

Embodiment 1. A method for isolation and detection of a single virus from a sample, comprising:
a) diluting the sample containing a virus;
b) encapsulating one virus from the sample in a single droplet of a liquid;
c) optionally, reverse transcribing a nucleic acid sequence of the virus to produce a cDNA sequence; and
d) submitting the nucleic acid sequence of the virus or the cDNA sequence derived from the nucleic acid sequence of the virus to nucleic acid amplification using a pair of oligonucleotide primers that amplify the nucleic acid sequence of the virus or the cDNA sequence derived from the nucleic acid sequence of the virus, yielding an amplified nucleic acid sequence.

Embodiment 2. The method of embodiment 1, further comprising adding a fluorescent oligonucleotide probe to the nucleic acid sequence of the virus or the cDNA sequence derived from the nucleic acid sequence of the virus in step (d), wherein the probe targets a site in the nucleic acid sequence of the virus or the cDNA sequence derived from the nucleic acid sequence of the virus; and
e) detecting the fluorescence of the fluorescent oligonucleotide probe that anneals to the nucleic acid sequence.

Embodiment 3. The method of embodiment 2, further comprising:
f) extracting the amplified nucleic acid sequence from the single droplet into a parallel flow of aqueous medium.

Embodiment 4. The method of embodiment 3, further comprising:
g) sequencing the amplified nucleic acid sequence.

Embodiment 5. The method of embodiment 4, wherein the sequencing comprises labeling the amplified nucleic acid sequence with a barcode.

Embodiment 6. The method of embodiment 1, wherein the amplified the nucleic acid sequence is about 500 bp to 2000 bp or about 1300 bp.

Embodiment 7. The method of embodiment 1, wherein the droplet is about 50 pL in volume.

Embodiment 8. The method of embodiment 1, further comprising adding a DNA polymerase, a buffer, magnesium chloride, and deoxynucleoside triphosphates (dNTPs) to the nucleic acid sequence of the virus or the cDNA sequence derived from the nucleic acid sequence of the virus in step (d).

Embodiment 9. The method of embodiment 1, wherein the sample containing the virus is diluted to less than about 10⁴ PFU/mL.

Embodiment 10. The method of embodiment 1, wherein the sample containing the virus is diluted into buffer.

Embodiment 11. The method of embodiment 1, wherein the liquid is deionized water or a buffer and a fluorinated oil.

Embodiment 12. A single-droplet extraction system comprising:
a) an optical droplet detector, comprising two laser sources and four mirrors focused into the microfluidic channel; two photomultiplier tubes (PMTs); and a high-speed camera for photoelectric converting and optical imaging;
b) a microfluidic device comprising a flow-focusing channel and a square chamber;
c) an electro-pneumatic valve; and
d) a rotation collection platform with a step motor fixed on the bottom of the rotation platform.

Embodiment 13. The system of embodiment 12, wherein the two PMTs are configured to receive a signal from fluorescent probe excited by the two laser sources.

Embodiment 14. The system of embodiment 12, wherein the electro-pneumatic valve is configured to actuate when a signal is received from the optical droplet detector.

Embodiment 15. The system of embodiment 12, wherein the lasers have a wavelength of about 488 nm.

Embodiment 16. The rotation collection platform system of embodiment 12, wherein the platform is configured to rotate upon receipt of a transistor-transistor logic (TTL) signal at a step angle of about 0.9°.

Embodiment 17. A microfluidic device comprising a flow-focusing channel and a square chamber, wherein the chamber has a height of about 45 μm.

Embodiment 18. The microfluidic device of embodiment 17, wherein the flow-focusing channel and the square chamber are polydimethylsiloxane (PDMS) or polymethyl methacrylate (PMMA) can also be used.

We claim:

1. A method for isolation and detection of a single virus from a sample, comprising:
a) diluting the sample containing a virus;
b) encapsulating one virus from the sample in a single droplet of a liquid;
c) optionally, reverse transcribing a nucleic acid sequence of the virus to produce a cDNA sequence; and
d) submitting the nucleic acid sequence of the virus or the cDNA sequence derived from the nucleic acid sequence of the virus to nucleic acid amplification using a pair of oligonucleotide primers that amplify the nucleic acid sequence of the virus or the cDNA sequence derived from the nucleic acid sequence of the virus, yielding an amplified nucleic acid sequence.

2. The method of claim 1, further comprising adding a fluorescent oligonucleotide probe to the nucleic acid sequence of the virus or the cDNA sequence derived from the nucleic acid sequence of the virus in step (d), wherein the probe targets a site in the nucleic acid sequence of the virus or the cDNA sequence derived from the nucleic acid sequence of the virus; and
e) detecting the fluorescence of the fluorescent oligonucleotide probe that anneals to the nucleic acid sequence.

3. The method of claim 2, further comprising:
f) extracting the amplified nucleic acid sequence from the single droplet into a parallel flow of aqueous medium.

4. The method of claim 3, further comprising:
g) sequencing the amplified nucleic acid sequence.

5. The method of claim 4, wherein the sequencing comprises labeling the amplified nucleic acid sequence with a barcode.

6. The method of claim 1, wherein the amplified the nucleic acid sequence is about 500 bp to 2000 bp or about 1300 bp.

7. The method of claim 1, wherein the droplet is about 50 pL in volume.

8. The method of claim 1, further comprising adding a DNA polymerase, a buffer, magnesium chloride, and deoxynucleoside triphosphates (dNTPs) to the nucleic acid sequence of the virus or the cDNA sequence derived from the nucleic acid sequence of the virus in step (d).

9. The method of claim 1, wherein the sample containing the virus is diluted to less than about 10⁴ PFU/mL.

10. The method of claim 1, wherein the sample containing the virus is diluted into buffer.

11. The method of claim 1, wherein the liquid is deionized water or a buffer and a fluorinated oil.

12. A single-droplet extraction system comprising:
a) an optical droplet detector, comprising two laser sources and four mirrors focused into the microfluidic channel; two photomultiplier tubes (PMTs); and a high-speed camera for photoelectric converting and optical imaging;
b) a microfluidic device comprising a flow-focusing channel and a square chamber;
c) an electro-pneumatic valve; and
d) a rotation collection platform with a step motor fixed on the bottom of the rotation platform.

13. The system of claim 12, wherein the two PMTs are configured to receive a signal from fluorescent probe excited by the two laser sources.

14. The system of claim 12, wherein the electro-pneumatic valve is configured to actuate when a signal is received from the optical droplet detector.

15. The system of claim 12, wherein the lasers have a wavelength of about 488 nm.

16. The system of claim 12, wherein the platform is configured to rotate upon receipt of a transistor-transistor logic (TTL) signal at a step angle of about 0.9°.

17. A microfluidic device comprising a flow-focusing channel and a square chamber, wherein the chamber has a height of about 45 μm.

18. The microfluidic device of claim 17, wherein the flow-focusing channel and the square chamber are polydimethylsiloxane (PDMS) or polymethyl methacrylate (PMMA).
